(19) United States
(12) Patent Application Publication
(10) Pub. No.: US 2016/0279391 A1
(43) Pub. Date: Sep. 29, 2016

(54) SOLID STATE METHODS FOR JOINING DISSIMILAR METAL GUIDEWIRE SEGMENTS WITHOUT THE USE OF TERTIARY MATERIAL

(71) Applicant: Lake Region Manufacturing, Inc., Chaska, MN (US)

(72) Inventors: Pankaj Gupta, Maple Grove, MN (US); Arne Rimmereide, Carver, MN (US); Anthony Hausladen, Lester Prairie, MN (US); James Allan Boegeman, Shakopee, MN (US)

(21) Appl. No.: 15/069,248

(22) Filed: Mar. 14, 2016

Related U.S. Application Data
(60) Provisional application No. 62/132,978, filed on Mar. 13, 2015.

(51) Int. Cl. A61M 25/09; C22C 19/03; B23K 20/00; B23K 20/227; B23K 20/02; B23K 20/12

(52) U.S. Cl. CPC A61M 25/09; B23K 20/02; B23K 20/129; B23K 20/002; B23K 20/227; C22C 19/03; B23K 2201/32

(57) ABSTRACT

A bi-metal medical guidewire with a core wire having a stainless steel proximal section and Nitinol distal section offers performance enhancements compared to guidewires made from either alloy alone. A solid-state, frictional welding or frictional joining process for Nitinol and stainless steel wires ranging in diameter from 0.013″ to 0.020″, without the need for tertiary metals, adhesive or ferrules is disclosed. The resulting frictional weld joint strength is approximately 80% of the tensile strength of the raw Nitinol wire with excellent bending properties.

### REFERENCE TO RELATED APPLICATION

This application claims the priority of and incorporates completely by reference (including its attachments), U.S. Provisional Patent Application Ser. No. 62/132,978, filed on Mar. 13, 2015.

### TECHNICAL FIELD

The present invention relates to the field of solid state material joining or fusing, particularly titanium, and titanium-based alloy workpieces, to ferrous metal workpieces e.g., by friction welding, without utilization of filler materials at their joint, interface, or juncture. The invention relates in particular without limitation to the field of medical devices such as guidewires.

### BACKGROUND OF THE INVENTION

A guidewire is a medical device used in various minimally invasive vascular applications. Its foundation is a metal core wire which is typically constructed of stainless steel or nitinol. A metal coil, a polymer jacket, or combination of the two, covers the core wire on its distal end, i.e., the end which interacts with a patient, in order to make the distal segment atraumatic, kink resistant, and flexible. For cost and performance reasons, ferrous metal, e.g., stainless steel, proximal segments or portions generally are used.

Designing a guidewire is an intricate exercise in balancing strength and flexibility. A guidewire with a spring temper stainless steel core has good pushability and torque transmission, owing to its high yield strength and Young's modulus. Those properties are important to the proper functioning of the guidewire in order to navigate to the desired vascular treatment site(s) and to deliver the intended diagnosis or therapeutic treatment. However, exceeding the yield strength of the guidewire core wire material in a bending mode results in permanent bends and kinks, severely reducing the guidewire performance. Nitinol is a super-elastic material providing great kink-resistance, but lacking pushability due to an inherent lower Young's modulus, resulting in less support in delivering therapy or devices. Ideally, a guidewire core combines the excellent mechanical properties of stainless steel in the main body, with the kink-resistance of Nitinol at the distal tip.

Metallurgically, joining e.g., nitinol to stainless steel, via prior art fusion welding processes is challenging due to the formation of brittle Fe—Ti intermetallics [1, 2]. One method of avoiding formation of such brittle intermetallics is to use a tertiary material (or “filler material” as it is sometimes referenced), such as nickel or tantalum when e.g., joining stainless steel to nitinol [3], use of a tertiary material, while sometimes necessary, tends to adds cost, weight and complexity to device design.

One method used in joining guidewire components is to insert the ends of the stainless steel and nitinol into a ferrule (i.e., a segment of what is referred to as hypotube) and then securing both ends using adhesive or solder. This method requires pre-processing to reduce the diameter at the ends of each core in order to fit the parts together, adding cost and complexity. This decrease in core diameter, along with the differing stiffness of the section of hypotube, and the addition of joint material, creates a kink point and may decrease clinical performance.

This invention is a solid-state weld process or fusion process to form device component interfaces such as butt joints between Nitinol and stainless steel wire or hypotube without the use of intermediary tertiary metals or tertiary materials. The process was used to join core wire segments having outside diameters ranging from 0.013″ to 0.020″. Larger and smaller diameter guidewire core wire segments are within the scope of this invention. In addition, complete 0.014″ outer diameter guidewires were built using the solid-state weld technology.

### BRIEF SUMMARY OF THE INVENTION

Briefly, on one aspect, a solid state method of joining e.g., by friction welding, titanium, and titanium-based alloys to ferrous metals is disclosed. In another aspect, the solid state intermetallic interfacial zone obtained in the aforementioned process also is disclosed. A solid state joining process, and the interfacial zone so generated, is free of filler materials known to the art. The instant invention, in a preferred aspect, involves friction welding of titanium-based alloys to ferrous metals, in the absence of filler materials deployed at the joint or zone, to create a surprisingly strong solid state intermetallic bond at the interfacial zone. Medical device applications such as guidewire components e.g., core wires and hypotube segments, and completed guidewire assemblies utilizing such components, are a particularly preferred application of this invention.

This invention is a guidewire comprising:

a core wire, the core wire comprising a titanium-based alloy segment; and

a ferrous metal segment, the titanium-based alloy core wire segment and the ferrous metal core wire segment each defining a frictional weld working surface, the frictional weld working surface of the titanium-based alloy core wire segment and the ferrous metal core wire segment being fused to each other at their working surfaces by a solid state frictional weld, the frictional weld being substantially free of tertiary materials.

Specific preferred features include:
The titanium-based alloy core wire segment comprises nitinol and the ferrous metal core wire segment comprises stainless steel.
A coil is disposed at least partially around the titanium-based alloy core wire segment (FIG. 3A).
The core wire segments have diameters in the range of 0.010″ to 0.040″.
The frictional weld has a fine-grain structure.
The frictional weld (HAZ) has fine grain structure grains which are no larger than the grain structure of the ferrous metal or titanium-based alloy segments and no substantial grain growth occurs due to the friction weld process.
The diameters of the titanium-based alloy core wire segment and the ferrous metal core wire segment at the frictional weld are all substantially the same.

The invention also includes a solid state method of fusing a guidewire titanium-based alloy core wire segment to a ferrous metal core wire segment comprising the steps of: providing a titanium-based alloy guidewire core wire segment having a frictional weld working surface; providing a ferrous metal guidewire core wire segment having a frictional weld working surface; and frictional welding the working surfaces of the core wire segments to each other in a solid state frictional welding step, the frictional welding step being free of the presence of a tertiary material.

The Specific preferred features include:
The frictional welding step is accomplished by rotating the working surfaces of the core wire segments with respect to each other at different speeds while applying force to the segments so as to create frictional heating.
The titanium-based alloy core wire segment comprises nitinol and the ferrous metal core wire segment comprises stainless steel.

The terms “titanium-based alloy” or “titanium-based system” as used herein are to be broadly construed to mean systems containing the two mentioned metals which exhibit the difficult bonding characteristics of nitinol. Titanium-based systems generally will contain significant atomic percentages of nickel with the titanium and nickel being present in an intermetallic phase. Without limitation, shape memory alloys as used in this invention, and as used to produce medical devices will consist essentially of about 48 to about 52 atomic percent nickel and conversely, from 52 to about 48 atomic percent titanium. Titanium-based alloys can and often do include additional elements such as copper, niobium, gold, palladium, platinum, hafnium and zirconium. The presence of any such third elements, according to this invention, is to be at a lesser atomic percentage than will materially inhibit the characteristic of shape memory effect in the resulting alloy or eutectic mixture. Alternative characterizations of materials to be included in this definition are those titanium-based alloys or eutectics which exhibit the phenomenon described as being “super elastic” at the temperature of their intended use.

The term “friction welding” (or its equivalent “frictional welding”) is used throughout this disclosure. Friction welding is a solid-state welding or joining process that generates heat through mechanical friction between workpieces in relative motion to one another, often with the application of force, to cause heating and fusion of the workpieces. Because no melt occurs, friction welding is not, strictly speaking, a welding process, but is a forging technique. However, due to the similarities between these techniques and traditional welding, the term “frictional welding” is used, recognizing that no weld, usually meaning fusion weld, is intended. “Frictional welding” as used herein is intended to include related solid state fusion processes not employing a filler material such as spin welding, linear friction welding, linear vibration welding, orbital friction welding, and inertial friction welding.

Frictional welding, in addition to overcoming the difficulties of joining titanium, and titanium-based alloys, to ferrous metals and ferrous-based alloys has other significant advantages over fusion welding techniques. The combination of fast joining times (on the order of a few seconds), and direct heat input at the weld interface, yields relatively small heat-affected zones. Friction welding techniques which are generally melt-free at the frictional weld interface avoid or reduce interfacial grain growth which can inhibit joint quality. Another advantage is that the frictional motion and optional application of pressure tends to “clean” the interface between the workpieces being joined, which means they can be joined with less preparation.

Further, frictional welding of titanium, and titanium-based alloys, to ferrous metals and ferrous-based alloys according to this invention is done without the need for (and in fact discouragement of) the interposition of a tertiary material or filler material between the workpieces to be frictionally joined. The advantages of that feature are discussed above.

The term “tertiary material” or its equivalent “filler materials” is used herein, see e.g., U.S. Pat. No. 6,875,949. Specifically, “tertiary material-free” or “filler material-free” and variations thereof are used. A tertiary material is any material not originating from the workpieces herein fused by friction welded; or which is created in the friction welding process itself. Fluxes, solder, other metals e.g., added Ni, Fe, or Ta, are examples of tertiary materials which in practice of this invention are absent from the weldment joint, weld zone, or frictional weld herein created.

### BRIEF DESCRIPTION OF THE DRAWINGS

Without limiting the scope of the present invention as claimed below and referring now to the drawings and figures.

FIGS. 1 and 2 show schematically an application of this invention. Specifically, FIG. 1 shows an elevational view of titanium, or titanium-based alloy workpiece 100, and a ferrous metal workpiece 200 in the embodiment of two adjacent wires, such as guidewire core wire segments (i.e., 100 and 200) with a joint precursor 300 between their ends;

FIGS. 3A, 3B, 3C and 3D show a completed guidewire distal assembly obtained by the use of the present invention and various core wire configurations commonly employed. FIGS. 3A, 3B, 3C and 3D also show a core wire friction weld zone or interface 400 as well as several NiTi/SS workpiece diameter relationships.

FIG. 4A is an SEM image of the joint post polishing illustrating the seamless nature of the joint.

FIG. 4B is an SEM image of the joint after etching showing the presence of small grains in HAZ.

FIGS. 5A and 5B are SEM images of fracture surface post tensile testing at low magnification (200×) of a hypotube joint and the present invention solid-state joint, respectively.

FIGS. 5C and 5D are SEM images of fracture surfaces post tensile testing as a high magnification image (2000×) of the NiTi wire side of a hypotube joint and the present invention solid-state joint, respectively.

FIGS. 6A and 6B show testing of a full guidewire 0.014″ diameter in 2-D plate model for a hypotube joint used in the prior art, and a solid-state weld joint of the invention.

FIG. 7 is a graph of the EDS analysis at the interface of the solid-state weld joint of Nitinol and stainless steel, collected within 10 μm on either side of the joint.

FIG. 8 is a graph showing that there is no statistically significant difference, at a 95% confidence level, in joint tensile strength with reduction in cross sectional area, which shows constancy of the joint.

FIG. 9 is a graph of the lateral stiffness on 0.014″ diameter guidewire with the present invention solid-state joint, in comparison to competitive guidewire with hypotube joint.

### DETAILED DESCRIPTION OF THE INVENTION

Titanium and titanium alloys have become important structural metals due to an unusual combination of properties. These alloys have strength comparable to many stainless steels at much lighter weight. Additionally, they display excellent corrosion resistance, superior to that of aluminum and sometimes greater than that of stainless steel. Further, titanium is one of the most abundant metals in the earth's crust and, as production methods become more economical, it will be employed in ever growing applications.

Various alloys of titanium and nickel are part of an alloy class known as shape memory alloys (SMAs). This term is applied to that group of metallic materials (also known as nickel-titanium alloys) that demonstrate the ability to return to a defined shape or size with thermal processing. In a most general sense, these materials can be plastically deformed at some relatively low temperature and return to their pre-deformation shape upon some exposure to higher temperatures. This shape memory effect (as it is sometimes called) i.e., the ability to exhibit a temperature dependent change in shape or configures, finds numerous commercial, especially medical, applications.

Nickel-titanium SMAs undergo a phase transformation in their crystalline structure when cooled through a transition temperature from the relatively stronger, high temperature or “austenite (or austenitic)” form to the relatively weaker, low temperature or “martensite (or martensitic)” form. Such crystalline transformations are responsible for the hallmark characteristics of these materials; their thermal, or shape memory; and their mechanical memory.

The characteristics of titanium and titanium-based alloys (conversely nickel-titanium alloys), especially their shape memory, means they have been widely used as components of medical devices such as catheters, stents, guidewires, blood filters, stylets, and numerous other devices.

A major limitation in the use of titanium and nickel-titanium alloys has been the difficulty of joining these materials to other materials. Because of its high cost, it is often desirable to limit the use of nickel-titanium to the actual moving parts of a device, while fabricating supporting members from less expensive materials such as stainless steel or other ferrous metals. However, conventional fusion welding of nickel-titanium to stainless steel and to ferrous metals in general has proven to be particularly difficult, as disclosed by Ge Wang, in a review “Welding of Nitinol to Stainless Steel”, in SMST-97: SMST-97: Proceedings of the Second International Conference on Shape Memory and Superelastic Technologies, ed. by Alan Pelton, et al, (SMST, 1997), pp. 131-136.

In addition, the reactivity of titanium makes it important that any welding be done in a clean, inert atmosphere e.g., as argon blanket, or in a vacuum, to reduce the tendency to form damaging oxides or nitrides. Nickel-titanium alloy materials naturally form surface oxides in air during processing into finished form making the use of an inert atmosphere (or vacuum) of lesser importance. The principal surface oxide formed is TiO₂.

Various methods have been used to attempt to improve results in joining of titanium alloys to ferrous metals and ferrous metal alloys. Those methods are variously described in the following United States patents which are incorporated by reference in their entireties herein: U.S. Pat. Nos. 4,674,675; 3,038,988; 4,708,282; 6,410,165; and 6,875,949. None of their approaches has been completely satisfactory.

The method of this invention is generally applicable to joining all titanium or titanium-based alloys (and conversely nickel-titanium alloys) workpieces and ferrous metal workpieces which can be oriented with respect to each other so as to create sufficient friction to generate heat (and potentially application of force), by the utilization of known frictional welders. Workpieces used in this invention will have frictional weld working surfaces at which friction surfaces will be created to generate a solid state (i.e., not a fusion weld) friction weld bond. Rotational frictional welding is a preferred frictional weld process in practice of this invention. The workpieces may be in any shape, including sheet, bar, tube, or, in the preferred embodiment, wire which meets the constraint that sufficient frictional interaction can be created to generate a solid state bond. Optional steps include cleaning and stress relieving the workpieces prior to joining, after joining, or both. Workpieces also may be resized e.g., by grinding, and the frictional weld workpiece interface treated to provide properties of interest.

These variations, modifications, alternatives, and alterations of the various preferred embodiments, processes, and methods may be used alone or in combination with one another as will become more readily apparent to those with skill in the art.

It is to be understood that the present invention while illustrated in the context of medical devices and device components, e.g., guidewires, is broadly applicable to the creation of any joint or weld involving titanium or titanium alloys and ferrous metals.

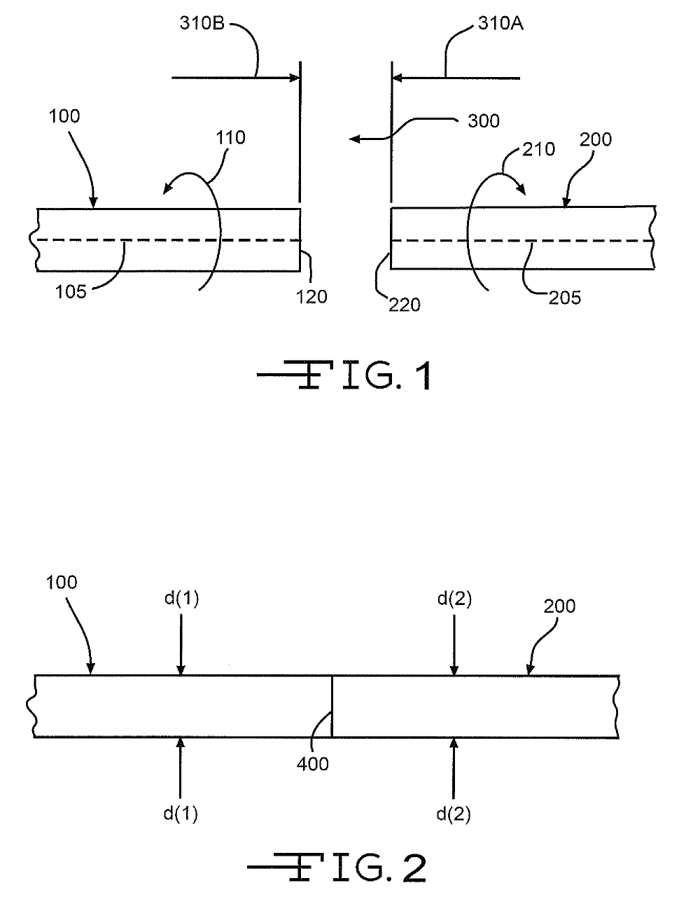

With reference generally now to FIGS. 1 and 2, the present method and friction welding interface are schematically shown. The method illustrated comprises rotational friction welding titanium, or a titanium-based alloy workpiece 100 directly to a ferrous metal workpiece 200 to produce a strong ductile interface, or fusion zone (workpieces 100, 200 provide what is sometimes referred to here as native workpiece material or native workpieces meaning the workpiece material(s) prior to friction welding and adjacent the frictional weld zone after welding.) Workpieces 100 and 200 define circular-in-section faces 120, 220 which are disposed substantially perpendicular to their respective axes of rotation 105, 205. Faces 120, 220 exemplify workpiece frictional welding working surfaces which are used to fuse the workpieces together in the preferred rotation frictional welding process. In general, the method involves aligning at least one titanium, or a titanium-based alloy, workpiece 100 in close proximity to at least one ferrous metal workpiece 200 (the designations may be interchanged) on a conventional friction welding apparatus (not shown) capable of rotating the two workpieces around axes of rotation 105 and 205 thereby forming a joint precursor 300. Arrows 310A and 310B show the axial direction workpieces 100 and 200 are moved toward each other by the friction welding apparatus as they are rotated about their axes of rotation 105 and 205 in the direction of arrows 110, 210 so as to frictionally interact either before, after, or simultaneously with their being rotated. The workpieces can be rotated in the same direction (110, 210), but with a differential of from about 5,000 rpm to about 50,000 rpm. The workpieces 100, 200 can also be rotated in opposite directions about their respective axes 105, 205. In this latter case, the differential in rpms need not be significant and counter rotation provides the requisite friction at an interface or friction weld zone 400.

Optionally, shielding is provided around the joint precursor, such as by way of example and not limitation, placing the workpieces 100, 200 in a vacuum or by flooding the joint precursor 300 and welding zone 400 with inert gas.

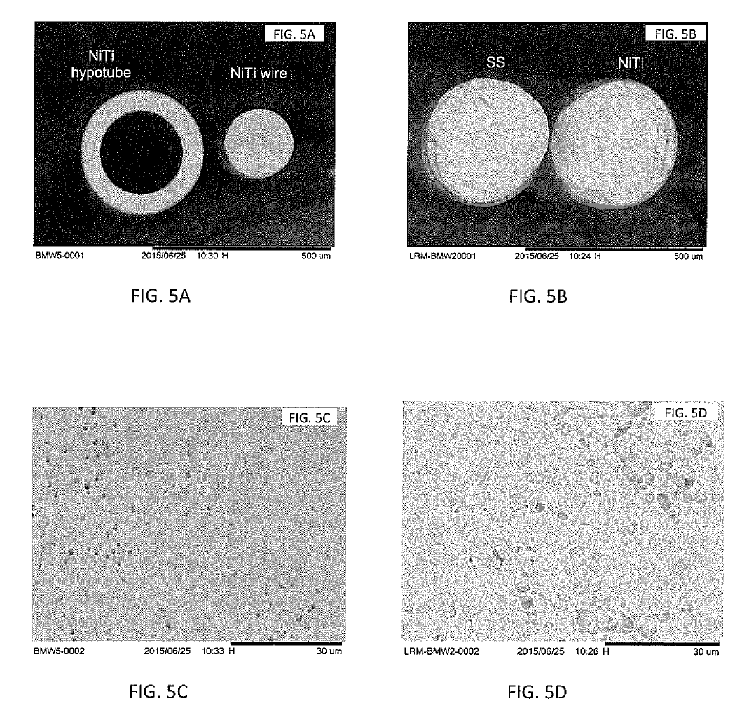

With reference now to FIG. 2, the joint precursor 300 then is closed by moving workpieces 100 and 200 toward each other so that their respective working surface ends or “butts” 120, 220 (as in butt weld) come into contact with each other at interface or frictional weld zone 400. Force in this range of about 10 pounds is applied which by the frictional welder creates (when the workpieces are rotated) a glow at interface 400 indicating creation of a plastic deformation state. It also is to be noted that core wire diameters d(1) and d(2) are substantially the same and correspond to the weld zone 400 diameter. Generally, no recrystallization is created at the weld zone. Further, it has been found that a very thin deformed zone e.g., 0.008″ to 0.015″ as formed in the native workpiece material 100, 200 on either side of the weld zone 400. Frictional welding then is undertaken by means of the frictional welding apparatus rotating one or the other or both of workpieces 100, 200 against each other (at different speeds) along their respective axes of rotation 110, 220 to interact, to create friction and heat and to cause solid state fusion. Force may be and often is applied from opposite sides of the joint (i.e., in the directions shown by arrows 310A and 310B) to heat the interface more rapidly. Obviously, one or the other or both of workpieces 100 and 200 can be rotating at the time they are forced against each other. One or the other of workpieces 100, 200 may be held stationary while rotating the other workpiece. Once the workpieces have been rotated with respect to each other so as to create a solid state joint, the joint will be allowed to “cool” or set to create the solid state zone, joint, weldment or “weld”. Exemplifying this schematic description of the invention, workpieces 100 and 200 can be, for example, guidewire core wire segments (shown in FIG. 5B), the resulting product being the core wire of a titanium alloy distal segment or tip guidewire core wire with no tertiary material or core wire segment. Workplaces 100 and 200 can also be hypotube segments (FIG. 5A) in which case an annular friction weld zone is created. FIG. 2 illustrates a portion of a guidewire core wire frictionally welded at interface, weld or zone 400. Zone 400 is free of filler material or tertiary material, all of the interface metals originating from workpieces 100, 200.

Numerous refinements and variations of the basic method are possible. While the method is generally applicable to all titanium, and titanium-based alloys, and ferrous metal combinations, in one particular embodiment the titanium or titanium-based alloy workpiece 100 is nickel-titanium (such as the well-known material nitinol) and the ferrous metal workpiece 200 may be stainless steel. The ferrous metal workpiece and titanium or titanium-based alloy workpieces, 100, 200 may be wire having the same or different diameters. Hypotube segments (sometimes referred to as microtubing) may be joined using the teaching of this invention.

To select one of the many combinations of workpiece materials, by way of example and not by way of limitation, the titanium, or the titanium-based alloy, wire 100 may be nickel-titanium, the ferrous metal wire 200 may be stainless steel. The titanium, or titanium alloy, workpiece 100 and the ferrous metal workpiece 200 may be in any form, such as by way of example and not limitation, ribbon, sheet, bar, tubing including microtubing, solid wire, stranded wire, braided wire, providing the workpieces can frictionally interact sufficient that frictional welding can be used to join the workpieces.

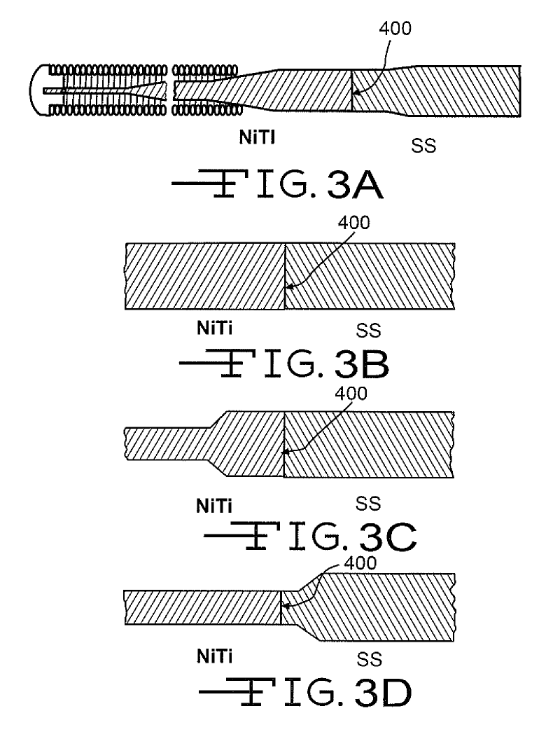

FIGS. 3A, 3B, 3C and 3D show a completed guidewire distal assembly obtained by the use of the present invention and various core wire configurations commonly employed. It is to be understood that the device shown in FIGS. 3A-3D is illustrative of one application of this invention; the invention is not to be construed as being limited thereto.

Numerous alterations, modifications, and variations of the preferred embodiments disclosed herein will be apparent to those skilled in the art and they are all anticipated and contemplated to be within the spirit and scope of the instant invention. For example, although specific embodiments have been described in detail, those with skill in the art will understand that the preceding embodiments and variations can be modified to incorporate various types of substitute, and/or additional or alternative materials, relative arrangement of elements, and dimensional configurations. Accordingly, even though only a few variations of the present invention are described herein, it is to be understood that the practice of such additional modifications and variations and the equivalents thereof, are within the spirit and scope of the invention as defined in the following claims.

The corresponding structures, materials, acts, and equivalents of all means or step plus function elements in the claims below are intended to include any structure, material, or acts for performing the functions in combination with other claimed elements as specifically claimed.

### Example and Analysis

Frictional weld solid-state joints using pre-straightened super-elastic binary Nitinol (54.5%-57.0% Ni) and 304v spring temper stainless steel wires with subsequent evaluation of joint strength, durability, and microstructure were evaluated. No tertiary materials or filler materials were interposed between the wire segments during the frictional fusing process. A conventional Aerotech PRO 225. Rotational Friction Welder was used in this work. Rotational speeds normally used in friction weld processes were used here. By way of example, rotational speeds in the range of 10,000 rpm to 40,000 rpm, preferably 25,000 rpm to 35,000 rpm were used.

The parts went through pre-conditioning, by cycling the joint ten times through a U-bend fixture with a radius of 0.1″ prior to obtaining tensile strength data by pulling the joint to failure using a MTS system. The joint microstructure was examined using standard metallographic methods of polishing and etching the longitudinal joint sections. In addition, optical microscopy and Scanning Electron Microscopy (SEM) confirmed the overall quality of the joint. Energy Dispersive Spectroscopy (EDS) analysis on the cross-section determined the weld zone length with intermixed Nitinol and stainless steel.

A grinding study was run on the frictional welded bi-metal joints using 0.018″ stainless steel to 0.020″ Nitinol wires. This allowed strength and joint quality evaluations closer to the core central axis. The wire, including the joint area, was ground to diameters of 0.014″, 0.010″, and 0.008″, then tensile tested to evaluate change in joint strength throughout the cross sectional area.

After initial joint strength and quality assessments, full guidewires were assembled using cores joined with the frictional weld process from 0.014″ stainless steel and 0.014″ Nitinol. The core wire distal grind profile for this study mimicked the stiffness profile of a commercially available bi-metal guidewire. The commercially available guidewire wire design consisted of Nitinol and stainless steel core wire segments joined via a Nitinol hypotube and glue. The two designs were tested side-by-side comparing lateral stiffness, tensile strength, and simulated clinical performance in a 2-D plate model emulating a tortuous vessel. After tensile testing, the fracture surface was analyzed using a table top SEM (Hitachi™ 300).

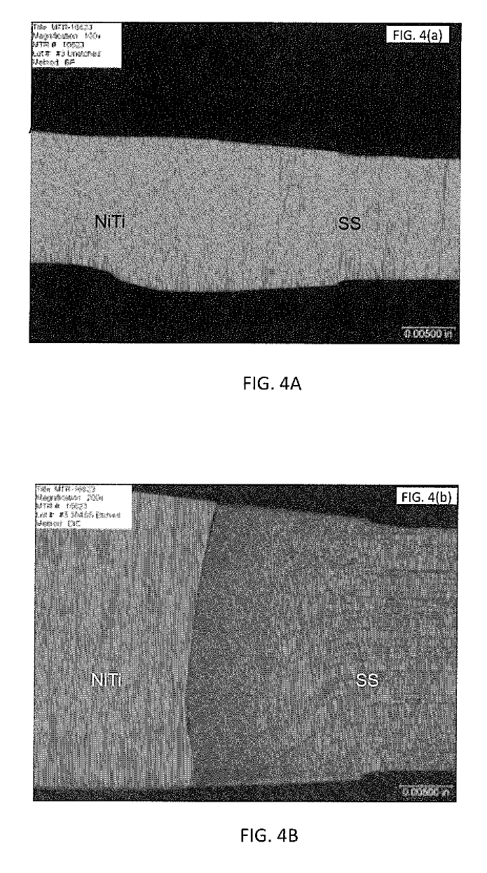

The results demonstrated that the solid state frictional welding process of this invention produces a clean and defined transition between the stainless steel and Nitinol and that the interface is free of defects and/or porosity. FIGS. 4A and 4B show SEM images of joint cross-sections between 0.018″ stainless steel and 0.020″ Nitinol. FIG. 4A shows the interface after polishing. FIG. 4B shows the interface after etching the stainless steel side. The heat-affected zone (HAZ) is approximately 0.012″ (˜300 micron) long, and is distinguished from the drawn wire elongated grain structure by the presence of a fine, uniform grain structure. A fine grain size is an inherent solid-state frictional welding advantage as compared to fusion welding processes, which is characterized by the presence of cast dendritic structure and large grains in the HAZ [2, 7].

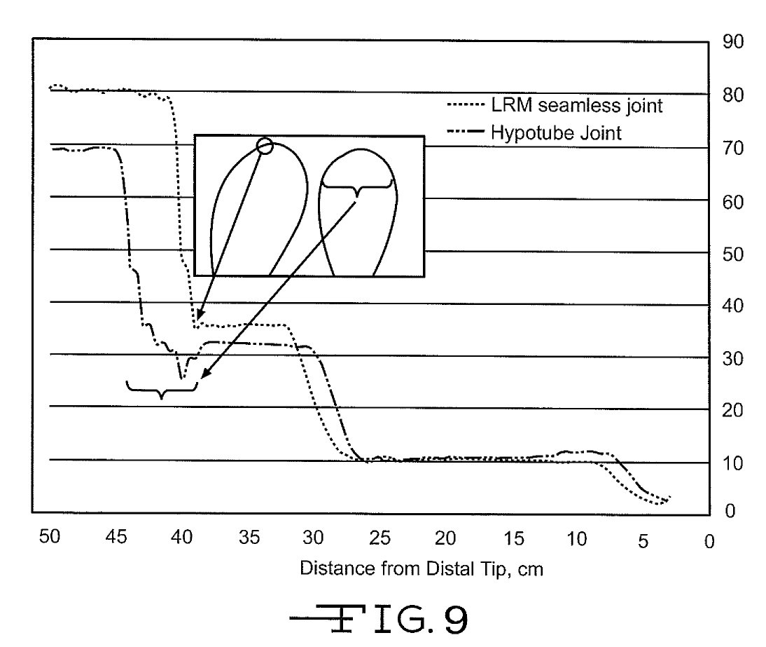

FIG. 7 shows the EDS analysis at the interface of the solid-state weld joint, collected within 10 μm on either side of the joint. The data shows that chemical intermixing of Nitinol and stainless steel extends approximately 1 μm on either side of the joint interface.

Stainless steel 0.018″ and 0.020″ Nitinol core wire segments or precursors were ground to different diameters post joining. The objective was to determine grindability of the joint, thus providing design options, and an assessment of joint strength uniformity toward the wire central axis. All the samples tested passed the U-bend pre-conditioning test, indicating excellent bending properties of the joint. The tensile test of the frictional welded joint yielded an average joint strength of approximately 80% of the tensile strength of Nitinol core wire, all failures occurring at the interface of the stainless steel and the Nitinol. This surprising and unexpectedly high joint strength and core wire versatility was hypothesized (without being bound to the hypothesis) to be due to the relatively small HAZ and the presence of fine grain structure contribute to the high strength of the joint.

With small sample sizes, the 95% confidence interval indicates no loss of stiffness as the core diameter is reduced via grinding, FIG. 8.

Full guidewires were built with the tertiary material-free frictional solid-state welded joints located approximately 40 cm from the distal tip. The grind profile and joint location selection aligned with leading competitive guidewires and enabled comparative bench testing. FIG. 9 shows the lateral stiffness results. The insert in FIG. 9 shows a frictional weld solid state joint in comparison to the hypotube joint design. It is evident that the joint of this invention is significantly shorter than the three-centimeter long hypotube joint. The solid-state weld also shows a smooth and even bending transition from stainless steel to Nitinol, while the hypotube joint design exhibits sharp transitions that could cause kinks and performance degradation.

At around 40 cm from distal tip, the lateral stiffness graph shows the seamless nature of the present invention guidewire by the direct change in stiffness at the solid-state weld joint. Conversely, the three-centimeter long hypotube joint shows an initial dip in the stiffness load prior to a more jagged increase.

Table 1 summarizes the tensile data for 0.014″ diameter frictional welded guidewires. The joints or interfaces exhibits high tensile strength compared to the hypotube glue.

TABLE 1
Tensile test results of present invention solid-state joint
compared to hypotube joint wire for wire diameter 0.014″

| | | | % of NiTi | |
|---|---|---|---|---|
| | No of | Break Load, | break | Failure |
| Sample ID | Sample | (Std Dev) Lbs | Load* | Location |
| Present | 5 | 28.6 (0.72) | 89 | At joint |
| Invention | | | | interface |
| Seamless | | | | |
| Joint | | | | |
| Hypotube | 5 | 5.1 (0.35) | 16 | NiTi wire |
| Joint | | | | attached to |
| | | | | the NiTi |
| | | | | hypotube |

*NiTi wire tensile strength for 0.014″ NiTi wire was about 32 lb

FIGS. 5A and 5C show that the failure mode for the hypotube joint design was adhesive failure, with subsequent core pullout from the hypotube. Therefore, the Nitinol wire end exhibits a smooth shear cut surface, FIG. 5C. The solid-state weld joint of this invention failed at or near the joint interface, FIG. 5B. The fracture surface of the invention solid-state weld exhibits micro-roughness and dimples on the Nitinol wire, typical of a ductile fracture mode, FIG. 5D.

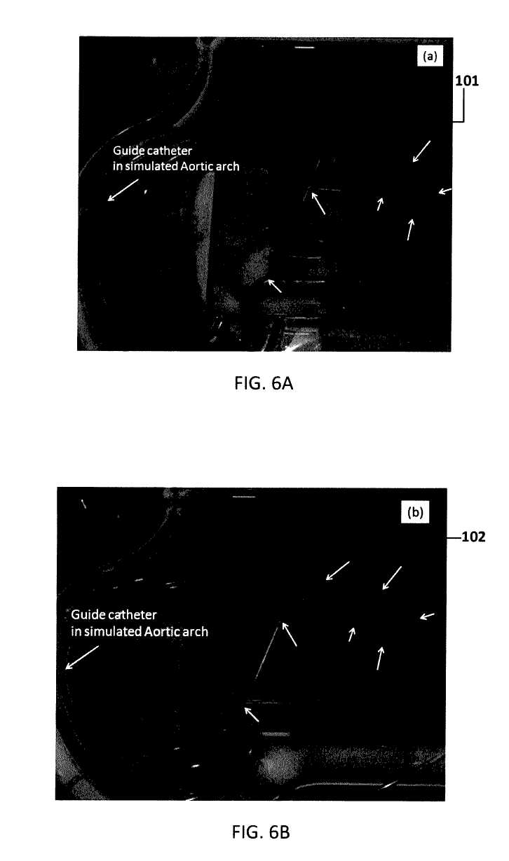

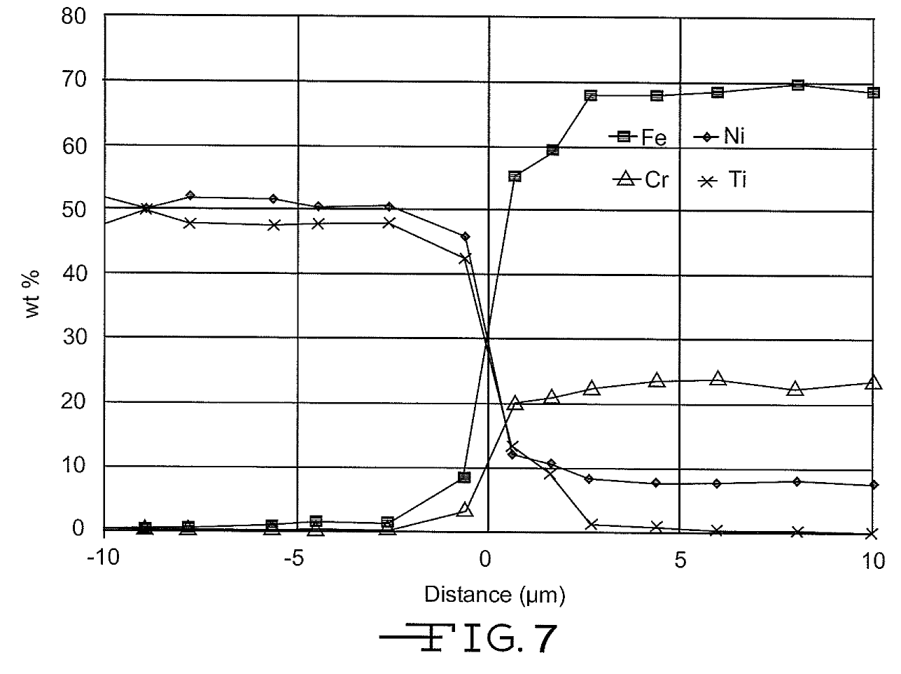

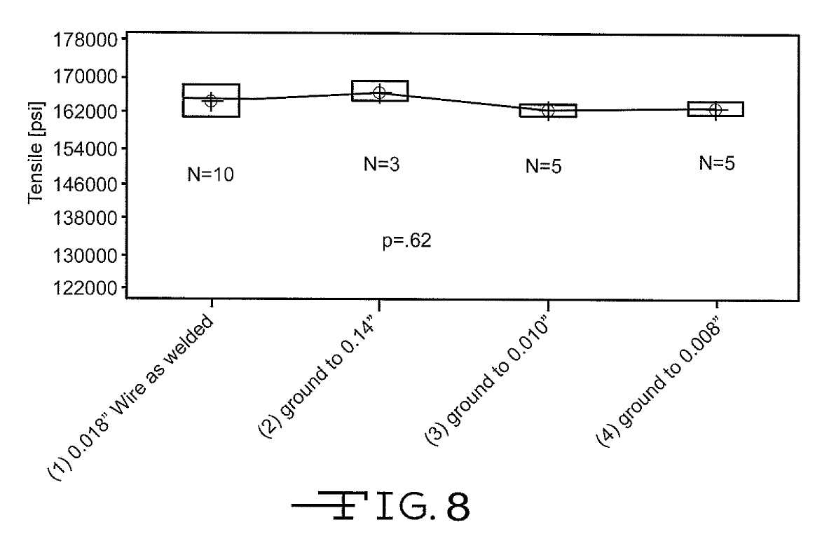

FIGS. 6A and 6B show the two wires simulated performance testing using a 2-D plate model. The 2-D plate model has several channels simulating tortuous vessels. The wire is inserted through a guide catheter (blue tube) into a predetermined pathway to assess tracking and torque response of the wire. The guidewire with the friction welded core wire was able to track much further into the pathway than the guidewire hypotube joint. Relatively lighter arrows 101 and 102 nearest the top of the FIGS. 6A and 6B, respectively, indicate the distal-most position that each wire navigated.

Nitinol core wire segments were joined to stainless steel core wire wires via frictional welding without the use of filler material. This process of this invention was shown to be an excellent method to create joints between dissimilar metals such as stainless steel and Nitinol. This process offers significant performance enhancements for guidewire applications, by merging a high stiffness stainless steel body, for pushability, with a softer, more kink resistant Nitinol, for the distal section. The solid-state frictional weld process yielded a fine-grained HAZ and a defect free interface resulting in excellent bend and tensile properties at the joint.

The initial performance testing using a 2-D plate model, (simulating vasculature), indicates that the present solid-state weld exhibits superior performance in a simulated clinical application compared to one of the leading competitive bi-metal guidewires in the market.

### REFERENCES

1. J. Pouquet, R. M. Miranda, L. Quintino, S. Williams, Dissimilar laser welding of NiTi to stainless steel, Int J. Adv Manuf Technol (2012), V61:205-212
2. P. Vondrous, L. kolarik and M. Kolarikova, Plasma Arc Welding of NiTi and 304 Steel, Annals of & Proceedings of DAAAM international, (2012) V 23, 1, 2304-1382
3. H. M. Li, D. Q. Sun, X. L. Cai, P. Dong and W. Q. Wang, Laser welding of TiNi Shape memory alloy and stainless steel using Ni interlayer, Materials & Design, (2012), V39 285-293
4. S. D. Meshram, T. Mohandas, G. M. Reddy, Friction welding of dissimilar pure metals, J Mat Proc Tech, (2007) V184 330-337
5. N. Kahraman, B. Gulenc & F. Findik, Joining of titanium/stainless steel by explosive welding and effect on interface, J. Mat. Processing Technol, (2005), V169, 127-133.
6. J. Tsujino, K. Hidai, A. Hasegawa, R. Kanai, H. Matsuura, K. Matsushima, T. Ueoka, Ultrasonic butt welding of aluminum, aluminum alloy and stainless steel plate specimens, Ultrasonics, (2002), 40 371-374.
7. A. Rajasekhar, Effect of welding process and post weld heat treatments on microstructure and mechanical properties of AISI 431 martensitic stainless steel, Int J. Tech. Research and Appl, (2015), V3: 280-285

What is claimed is as follows:

1. A guidewire, comprising:
a) a core wire, the core wire comprising:
i) a titanium-based alloy segment; and
ii) a ferrous metal segment, the titanium-based alloy core wire segment and the ferrous metal core wire segment each defining a frictional weld working surface,
b) wherein the frictional weld working surface of the titanium-based alloy core wire segment and the ferrous metal core wire segment are fused to each other by a solid state frictional weld, the frictional weld being substantially free of tertiary materials.

2. The guidewire of claim 1 wherein the titanium-based alloy core wire segment comprises nitinol and the ferrous metal core wire segment comprises stainless steel.

3. The guidewire of claim 1 wherein a coil is disposed at least partially around the titanium-based alloy core wire segment.

4. The guidewire according to claim 1 wherein the core wire segments have diameters in the range of 0.010″ to 0.040″.

5. The guidewire according to claim 1 wherein the frictional weld has a fine-grain structure.

6. The guidewire according to claim 5 wherein the grain structure is no larger than the grain structure of the ferrous metal or titanium-based alloy segments.

7. The guidewire according to claim 1 wherein the diameters of the titanium-based alloy core wire segment and the ferrous metal core wire segment are substantially the same.

8. A solid state method of fusing a guidewire titanium-based alloy core wire segment to a ferrous metal core wire segment, comprising the steps of:
a) providing a titanium-based alloy guidewire core wire segment having a frictional weld working surface;
b) providing a ferrous metal guidewire core wire segment having a frictional weld working surface; and
c) frictionally welding the working surfaces of the core wire segments to each other in a solid state frictional welding step, the frictional welding step being free of the presence of a tertiary material.

9. The method according to claim 8 wherein the frictional welding step is accomplished by rotating the working surfaces of the core wire segments with respect to each other at different speeds while applying force to the segments so as to create frictional heating and once solid state bonding has occurred between the working surfaces, permitting the working surfaces to cool.

10. The process according to claim 8 wherein the titanium-based alloy core wire segment comprises nitinol and the ferrous metal core wire segment comprises stainless steel.

11. The process of claim 8 including rotating the titanium-based alloy guidewire core wire segment and the ferrous metal guidewire core wire segment same direction.

12. The process of claim 11 including rotating the titanium-based alloy guidewire core wire segment and the ferrous metal guidewire core wire segment with a differential of from about 5,000 rpm to about 50,000 rpm.

13. The process of claim 8 including rotating the titanium-based alloy guidewire core wire segment and the ferrous metal guidewire core wire segment in opposite directions.

14. A frictional weld or weld zone created according to the process of claim 8.
